(19) United States
(12) Patent Application Publication
(10) Pub. No.: US 2025/0235206 A1
(43) Pub. Date: Jul. 24, 2025

(54) ROBOTIC SEWING SYSTEM AND METHOD OF USE

(71) Applicant: LEM Surgical AG, Bern (CH)

(72) Inventor: Yossi BAR, Muri bei Bern (CH)

(73) Assignee: LEM Surgical AG, Bern (CH)

(21) Appl. No.: 19/173,644

(22) Filed: Apr. 8, 2025

Related U.S. Application Data
(63) Continuation-in-part of application No. PCT/IB2023/055439, filed on May 26, 2023.
(60) Provisional application No. 63/634,161, filed on Apr. 15, 2024.
(60) Provisional application No. 63/418,675, filed on Oct. 24, 2022.

(51) Int. Cl. A61B 17/04; A61B 1/00; A61B 1/317; A61B 34/20; A61B 34/32; A61B 34/37; A61B 50/13; A61B 90/00

(52) U.S. Cl. CPC A61B 17/0491; A61B 1/00149; A61B 1/317; A61B 17/0469; A61B 34/32; A61B 34/37; A61B 50/13; A61B 2034/2055; A61B 2090/395; A61B 2090/3991

(57) ABSTRACT

A centrally coordinated and synchronized robotic system for robotic sewing procedures, such as bilateral spinal robotic sewing procedures, includes multiple robotic arms and a controller that can manipulate the robotic arms to position tools, end effectors, cameras, or navigation elements, such as a suturing needle and a marker or label for use in a robotic sewing procedure. The markers or labels may be placed on different tissue portions, including for example skin and surgically exposed tissue, to help guide the robotic arms and deployment of the markers to perform bilateral robotic sewing.

### CROSS-REFERENCE TO RELATED APPLICATIONS

This application claims the benefit of U.S. Provisional Application No. 63/634,161 (Attorney Docket No. 67551-710.102), filed on Apr. 15, 2024, and is a continuation-in-part of PCT Application No. PCT/IB2023/055439 (Attorney Docket No. 67551-710.601). filed on May 26, 2023, which claimed the benefit of U.S. Provisional Patent Application No. 63/418,675 (Attorney Docket No. 67551-710.101). filed on Oct. 24, 2022. the full disclosures of which are incorporated herein by reference.

### BACKGROUND

### Field

The disclosed technology relates to systems for robotically controlled and coordinated surgical procedures. In particular, the technology relates to robotic systems and methods intended to assist in or automatically perform robotic surgical suturing.

Robot-assisted surgery, commonly referred to as robotic surgery, refers to any type of open, minimally invasive, endoscopic, percutaneous vascular, or other surgery that is performed using a surgical robot. Surgical robots can take many different forms and can be configured to manipulate many different types of surgical tools and other objects, depending on the surgical procedure being performed.

Of particular relevance to the disclosed technology, surgical robots have been used to suture tissue as part of both open surgical and minimally invasive surgical robotic procedures, including spinal and other orthopedic procedures, bronchial procedures, urological procedures, gynecological procedures, and the like. In open robotic surgical procedures, the suturing is often performed by a user who “remotely manipulates” a suturing needle using one or two robotic arms while observing the surgical space on a display. It has also been proposed to allow a surgical robot to “automatically” control the robot arm(s) and, in some cases, specialized suturing tools to suture tissue according to specialized control algorithms while optically viewing with cameras and/or other sensors.

Remote manipulation of a suturing needle by a user has at least two drawbacks. First, direct human control is difficult and can be exhausting for the user, sometimes leading to mistakes. Second, even very skilled users can cause misalignment and “bunching” of the coapted edges of the tissue being sutured. Such misalignment can increase scarring after healing and in some cases compromise the closure.

Many surgical operations, such as spinal and other orthopedic surgeries, require the cutting and suturing of major muscles in a patient's back which can require a significant force (e.g. higher than a 3 kilograms force (kgf)) to advance the suturing needle and wire. Currently available robotic suturing devices often are too weak to handle suturing of major muscles, particularly over extended time periods.

Suturing the incision in a patient's back after spine surgery can require 40 minutes or more of exhausting, repetitive work with application of high forces, all of which is required after several hours performing a complex surgical procedure, sometimes resulting in less experienced staff members performing some or all of the suturing.

For these reasons, there is a strong felt need for a robotic system and method which is capable of assisting in and/or automatically performing robotic sewing of human tissue including both skin and underlying tissue (e.g., in back, legs, shoulders) and to be able to handle the required high forces while providing fully robotic closed-loop operation for robotic full or partial control of the surgical procedure. At least some of these objectives will be met by the technologies disclosed herein.

### Background Art

US Patent Publication Nos. US2023/0171147; US2020/0015806; US2008/0215065; and US2008/0177281; and PCT Publication Nos. WO2024/069345; WO2022/212284; WO2022/036152; WO2009/123891 and WO2000/30548, describe surgical robots having suturing capabilities. Commonly owned publications and applications include WO2022/195460; WO2023/067415; WO2023/118984; WO2023/118985; WO2023/144602; WO2023/152561; WO2023/223215; WO2023/237922; PCT/IB2023/055439; PCT/IB2023/056911; PCT/IB2023/055662; PCT/IB2023/055663; U.S. 63/524,911; and U.S. 63/532,753, the full disclosures of which are incorporated herein by reference.

### SUMMARY

In a first aspect, the disclosed technology provides a surgical robotic system for sewing an incision in a patient's soft tissue. An exemplary system comprises a first surgical robotic arm configured to carry a first tool adapted to grasp and release a surgical needle and a second surgical robotic arm configured to carry a second tool adapted to grasp and release the surgical needle. A third surgical robotic arm configured to position a sensor adapted to track positions of a plurality of markers placed on patient tissue on each side of the incision may also be included. The surgical robotic system further includes a surgical robotic controller configured to (a) either automatically or in response to user input control movements of the first and second surgical arms to pass the surgical needle through opposite edges of the incision and to tension a suture carried by the surgical needle to close the incision and (b) automatically adjust movements of the first and second surgical arms in response to changes in tissue marker positions detected by the sensor to properly align the opposite edges of the incision as the incision is being closed.

The surgical robotic controller will be configured to control movement of the surgical robotic arms; to send and receive data to and from the surgical robot, such as receiving image and other data from cameras or other sensors, receiving force and other operational data from the surgical robotic arms; and the like. User inputs may be provided to the surgical robot controller either remotely from a separate control station and/or from an interface mounted on or adjacent to the surgical robot. The surgical robotic controller will in some instances be capable of automatic, often autonomous, control of some or all robotic functions, while in other instances will respond to user input to directly control robotic arm placement and tool operation, allowing a user to have primary control over the robotic arms and/or surgical tools during all or a portion of the robotic surgical procedure. In all instances, the surgical robotic controllers of the disclosed technologies will be able to “adjust” particular automatic or user-controlled movements of the surgical robotic arms in response to observed changes of the tissue marker positions in order to properly align the edges of the incision as the edges are being brought together by the surgical robotic arms.

Use of the surgical robotic arms for direct movement of a surgical needle (in contrast to using a robotically located but separate sewing apparatus), is advantageous as the robotic arms are capable of exerting the large forces necessary to suture major muscles, such as those in a patient's back, which can require a significant force (e.g. higher than a 3 kilograms force (kgf)) to advance the suturing needle.

In some instances, the surgical robotic controller adjusts movement of the first and second surgical arms to draw markers disposed on opposite sides of the incision into a predetermined alignment as the incision is being sewn closed, for example, by drawing pre-positioned marker pairs into lateral alignment on opposite sides of the incision.

In some instances, the surgical robotic controller may be configured to adjust movements of the first and second robotic arms which have been directly input by a user. For example, the surgical robotic controller may be configured to (a) allow a user to directly control movements of the first and second surgical robotic arms to pass a needle through the soft tissue edges adjacent an incision and (b) automatically adjust movements of the first and second surgical robotic arms when pulling the suture to close the edges of the incision together.

In some instances, the surgical robotic controller may be configured to automatically position the third surgical robotic arm configured to position the sensor to track the tissue markers and to avoid interference with the first and second surgical robotic arms.

In some instances, the first and second tools may comprise needle holders having jaws controlled by the robotic surgical controller for grasping and releasing the surgical needle.

In some instances, at least some of the markers may be located on the patient's skin.

In some instances, at least some of the markers may be located on the patient's muscle exposed by the incision.

In some instances, at least some of the markers may be located on the patient's fascia exposed by the incision.

In some instances, the sensor may be a camera and the markers may be optically visible to the camera.

In some instances, the incision may be disposed in the patient's back to expose the patient's spine.

In some instances, all surgical arms may be mounted on a common chassis. For example, the chassis may comprise a surgical bed frame. Alternatively, the chassis may comprise a mobile cart, and the mobile cart may be configured to extend beneath and across a full width of a surgical bed to position the first and second robotic surgical arms on opposite sides of the bed.

In a second aspect, the disclosed technology provides a method for sewing (closing) an incision in a patient's soft tissue using a surgical robot having a surgical robotic controller. The surgical robotic controller controls movement of the first and second surgical robotic arms, with user input or automatically, to pass a surgical needle through opposite edges of the incision, wherein the surgical needle pulls a suture across the incision. The surgical robotic controller further controls movement of the first and second surgical robotic arms, with user input or automatically, to tension the suture to close the opposite edges together. The positions of a plurality of markers placed on patient tissue on each side of the incision are automatically tracked as the suture is being tensioned, and movements of the first and second surgical arms are automatically adjusted in response to changes in tissue marker positions to properly align the opposite edges of the incision as the suture is being tensioned.

In some instances, controlling movement of the first and second surgical robotic arms to pass a surgical needle through opposite edges of the incision comprises the user inputting instructions to the surgical robotic controller via an interface to guide movement of the first and second surgical robotic arms.

In some instances, controlling movement of the first and second surgical robotic arms to tension the suture to close the opposite edges together comprises the user inputting instructions to the surgical robotic controller via an interface to guide movement of the first and second surgical robotic arms.

In some instances, at least some of the plurality of markers are located on the patient's skin on either side of the incision.

In some instances, at least some of the plurality of markers are located on opposed surfaces of the patient's soft tissue exposed by the incision.

In some instances, at least some of the plurality of markers are located on opposed surfaces of the patient's fascia exposed by the incision.

In some instances, at least some of the plurality of markers are placed on the patient's skin prior to making the incision.

In some instances, at least some of the plurality of markers are placed on the patient's skin after making the incision.

In some instances, at least some of the plurality of markers are two-dimensional.

In some instances, at least some of the two-dimensional markers comprise flexible stickers or a flexible ribbon that can be adhesively attached to the patient tissue.

In some instances, at least some of the two-dimensional markers are marked on the patient's tissue with an ink.

In some instances, the user manually marks the patient's tissue with a marking pen.

In some instances, the surgical robotic controller positions a surgical robotic arm to mark the patient's tissue with the ink.

In some instances, at least some of the plurality of markers are three-dimensional.

In some instances, the method of the disclosed technologies further comprises tracking the positions of the plurality of markers placed on patient tissue on each side of the incision automatically as the surgical needle is passed through the opposite edges and adjusting movements of the first and second surgical arms automatically in response to changes in tissue marker positions to properly align the opposite edges of the incision as the surgical needle is passed through said opposite edges.

In some instances, tracking the positions of the plurality of markers placed on patient tissue on each side of the incision comprises scanning the tissue with a sensor controlled by the surgical robotic controller.

In some instances, scanning the tissue with a sensor controlled by the surgical robotic controller comprises controlling movement of a third surgical robotic arm to position a camera held by the third surgical robotic arm to optically scan the plurality of markers.

In some instances, tracking the positions of the plurality of markers placed on patient tissue on each side of the incision comprises scanning the tissue with a sensor controlled by the surgical robotic controller prior to tensioning the suture to close the opposite edges together to establish a baseline marker pattern from which changes can be determined.

In some instances, tracking the positions of the plurality of markers placed on patient tissue on each side of the incision comprises scanning the tissue with a sensor controlled by the surgical robotic controller prior to passing the surgical needle through opposite edges of the incision to establish a baseline marker pattern from which changes can be determined.

In some instances, tracking the positions of the plurality of markers placed on patient tissue on each side of the incision comprises scanning the tissue with a sensor controlled by the surgical robotic controller prior to forming the incision to establish a baseline marker pattern from which changes can be determined.

In some instances, the method of the disclosed technologies further comprises scanning the patient's tissue with the sensor after the suture has been tensioned to confirm that the markers on the opposite edges of the incision remain properly aligned.

In still other aspects of the disclosed technologies provides a mobile bilateral robotically controlled surgical system. Specifically, the disclosed system is a centrally coordinated and synchronized robotic system for robotic surgical procedures, optionally being optimized for robotic surgical procedures requiring the application of high forces while still providing excellent accuracy, such as the sewing of multiple tissue layers in spinal surgery or other orthopedic surgery. The system comprises multiple robotic arms that each can hold, place and/or manipulate multiple end effectors, camera/sensor elements or navigation/tracking elements for use in robotic surgical procedures. The end effectors may include any surgical tools useful for performing robotic surgical procedures. The cameras/sensors and navigation/tracking elements (including, but not limited to, markers, force/torque sensing, CT, MRI, navigation cameras, endoscopic cameras etc.) are for providing guidance for the movement of the robotic arms and deployment of end effectors and tools.

In some embodiments, a bilateral robotically controlled surgical system according to the disclosed technology may have two or more surgical arms capable of positioning end effectors and holding tools, along with one or more imaging, navigation or surveillance arms holding a navigation and/or endoscopic camera, with all of these arms being based on a single cart with a central control unit. The bilateral nature of the disclosed systems means that the single cart fits under a surgical table and at least one of the surgical arms extends upwards from under the surgical table on one side of the table and at least one of the surgical arms extends upwards from under the table on the other side of the table. The surveillance, navigation or imaging arm holds the navigation camera or in another embodiment, an endoscopic camera in an advantageous position for viewing the surgical field and any associated navigation/endoscopic cameras.

In some embodiments, a bilateral robotically controlled surgical system according to the disclosed technology may have four or more surgical arms capable of positioning end effectors and holding tools, along with two or more navigation or surveillance arms holding a navigation and/or endoscopic cameras. In these embodiments, at least two of the surgical arms and at least one of the surveillance arms are positioned on one mobile cart and at least two additional surgical arms and at least one additional surveillance arm are positioned on another mobile cart. The two carts may be joined together with appropriate mechanical and electrical connections such that one mobile unit is formed with a single control unit providing full robotic and navigation control. The joined mobile unit has at least four surgical arms and at least two surveillance arms and is capable of performing a wide range of surgical procedures while all the robotic arms are synchronized by the fact that they share the same mechanical chassis.

In the various provided embodiments of a bilateral robotically controlled surgical system, the origin of each of the surgical arms is positioned at least 80 cm apart from the origin of any of the other surgical arms, and in some cases at least one meter apart. This provides for full reachability, maneuverability, and control without kinematic constraint since the base of the arms are spaced far apart.

The disclosed technology describes a mobile bilateral surgical robotic system which may involve navigational markers and sensors used to robotically utilize bilateral synchronized surgical techniques. More particularly, described is a surgical technique focused on a soft tissue sewing apparatus. In the disclosed technology, at least two robotic arms, which are robotically synchronized, assembled and calibrated on a single rigid chassis are cooperating with at least one additional robotic arm which comprises at least one camera/sensor to keep track of the surgical procedure and the various markers and sensors used to perform the procedure. Additional robotic arms deployed on the single rigid chassis can be used to position and operate additional surgical tools and camera/sensor elements as desired.

As described, the disclosed technology comprises multiple robotic arms which access and visualize the surgical field in an automatic and safe way because they are robotically synchronized. In one embodiment, there may be three robotic arms, two of which place, guide and/or hold robotic tools and one holding a navigation camera. In such an embodiment, the first arm may optionally position and then control the use of, for example, a sewing tool such as a suturing apparatus. The second arm may optionally position and then control the use of, for example, a forceps or grasping tool. A third arm may optionally hold a camera/sensor that provides an image of the process from an optimal distance and angulation. The camera is able to operate from optimal distance and angulation because it is sized appropriately and is deployed on an appropriately sized and positioned robotic arm. Optionally, the robotic arms may also hold additional imaging or navigation cameras to provide redundancy and diversity of information. Also optionally, active or passive markers may be placed on various tissue elements inside the body or outside on the skin surface for example to assist the robotic system in positioning the robotic arms and surgical tools. Also optionally, the robotic arms and/or the end effectors may have active or passive markers placed on them that may assist the robotic system in positioning the robotic arms and/or the end effectors.

In one embodiment, the synchronized movement of the robotic arms is enabled by the interaction of the navigation cameras with active or passive markers that are placed on portions of the patient's anatomy and/or the robotic arms or end effectors. The movement of the robotic arms is synchronized by a central control unit from a single base that knows where the arms are based upon factory assembly and calibration. The navigation information provided by the various markers and the one or more cameras is supplementary and may assist during the procedure to add more information about the location of the arms in relation to the environment. The fact that all the robotic arms are assembled on a joined mechanical chassis and calibrated together frees the need to continuously monitor the markers on the robotic arms and tools and focus only on the markers placed on the tissue. This can contribute tremendously to the enablement of this robotic sewing procedure since it is practically impossible in many cases for the navigation/endoscopic camera to continuously track so many markers, especially when in many applications the end effectors are far apart from each other and their markers are obscured.

In one embodiment, the disclosed technology suggests putting on the outer surface of the muscles and/or skin and/or on the inner portion of the muscles and/or on certain internal organs markers with specific patterns (can be glued as stickers but can also be added in other manners such as sewed, stuck with a small needle etc.). The markers are seen and recognized by the camera/sensor and can be deployed on both sides of an incision on the muscles and/or skin so during the sewing procedure the camera recognizes them and can keep track of the changing relative positions between the various tissue components. In this way, while the robotic arms are sewing the patient, pushing the sewing needles inside, and pulling the needle and wires and by that deflecting and manipulating the soft tissue, the camera/sensor can keep track over the changing soft tissue and react accordingly. The sewing patterns can be detected by the human doctor intraoperatively and to be executed partially or fully by the robotic system or can be fully automatic being calculated and decided by Artificial intelligence (AI) based software (SW).

In various embodiments, the surgical arms are robotically synchronized so that they can work in synchronization with each other and in relation to the marked soft tissue. The robotic arms are equipped with force and torque sensors so they can sense forces and moments applied by the soft tissue and/or each other while working synchronously and exchanging tools etc. The robotic arms have the ability to be deployed bilaterally to each side of the patient's body or specific organ thus providing the advantage of working synchronously in relation to each other (similar to human work with 2 arms) and for providing appropriate reach and access to the surgical field for the desired application. The disclosed robotic arms, being robotically coordinated and controlled arms and not remote manipulators, are strong and sturdy and so can push and pull the needle and wire with the required forces (e.g. above 3 kgf) to firmly tighten together the sewed muscles of the back or other area of multi-layered tissue. Moreover, in this disclosure, the robotic arm bases are located at a distance from each other (e.g. at least 80 cm or 1 meter apart) which provides superior reachability and application of force/moments.

Accordingly, in various embodiments of the disclosed system and methods, passive or active markers may be used to assist in navigation during a robotic sewing procedure that may employ a bilateral approach. These procedures may require the placement of multiple passive or active markers on the patient's anatomy (on various tissue layers) or on the robotic arms, end effectors or tools. In particular embodiments, miniature markers may be preferred. Portions of the patient's anatomy, such as multiple skin layers, may be relatively small and positioned closely together, and so to place multiple markers on different anatomical portions, it may be advantageous to use relatively small markers (2 cm or less in size). When using small markers, it may be advantageous to have the one or more cameras be deployed quite close to the surgical field on multiple robotic arms, for example at a distance of 30 cm or less from the surgical field, and also at an advantageous angulation relative to the surgical field so that the marker(s) can be visualized. It may also be advantageous to place smaller markers on the robotic arms, end effectors or tools so that they do not obscure each other or aspects of the surgical field. This arrangement can then provide appropriate navigation information to the central control unit and provide for coordinated movement of the robotic arms in their positioning and operation of end effectors and surgical tools. To further explain, from such disclosed close proximity between the camera/sensor and the miniature marker and the required large number of markers on the anatomy and the robots and their end effectors and tools it is extremely advantageous not to need to observe all markers together but to be able to focus only on the anatomy markers. With the novel suggested robotic system this is achievable.

All of these needs and elements benefit tremendously from the central coordination and synchronized control of the disclosed single-cart, multi-arm, non-teleoperated robotic system. Based on the placement of appropriately sized markers and the placement of navigation cameras at an appropriate distance and orientation to the target anatomy and the markers, movement of the robotic arms carrying end effectors and cameras can be coordinated to provide for a safe and accurate robotic sewing procedure that applies required forces for the chosen application. Robotic full or partial control is also provided to alleviate surgeon exhaustion and to provide enhanced accuracy beyond human capability.

Many of the embodiments herein are described in connection with robotically coordinated sewing in robotic spinal surgery. However, any complex sewing task will benefit from the system and methods of the disclosed technology. The disclosed systems provide full bilateral reachability and robotic control without kinematic constraint and so accuracy will be enhanced in any sewing task when compared with teleoperated systems or single arm robotic systems.

### BRIEF DESCRIPTION OF THE DRAWINGS

FIG. 1 shows a surgical robot having needle holders and on-board imaging configured to perform the methods of the disclosed technology.

FIG. 2 illustrates the surgical robot of FIG. 1 positioned under a surgical bed while closing a midline incision on a patient's back (B), in accordance with the principles of the disclosed technology.

FIGS. 3A to 3D show an example of how markers may be placed on a patient's back when closing a midline incision on a patient's back (B) in accordance with the principles of the disclosed technology.

FIGS. 4A and 4B are detailed views showing needle and suture advancement while closing a midline incision on a patient's back (B) in accordance with the principles of the disclosed technology.

### DETAILED DESCRIPTION OF THE DISCLOSED TECHNOLOGY

With reference now to the figures and representative embodiments of the disclosed technologies, the following detailed description is provided.

With reference to the attached figures, the disclosed surgical robotic systems comprise at least a first surgical robotic arm for manipulating a needle holder and at least one additional surgical robotic arm for manipulating at least one camera or other sensor. Usually, the disclosed surgical robotic systems will further comprise a second surgical robotic arm for manipulating the needle holder with a third surgical robotic arm for holding the camera or other sensor. Additional arms for other purposes may also be provided. The surgical robotic arms may be “dedicated” arms with the first and second surgical robotic arms configured specifically to hold or incorporate the needle holders and the third surgical robotic arm configured specifically to hold or incorporate the camera or sensor. Alternatively, the surgical robotic systems may have two, three, four, or more “general purpose” surgical robotic arms which can interchangeably hold a variety of tools, sensors, and the like, including needle holders, cameras, and sensors, as part of the disclosed technologies.

The needle holders will be configured to be fixedly or interchangeably mounted at the distal ends of the first and second surgical robotic arms and will be able to releasably grasp conventional surgical needles for drawing wire and other sutures for sewing and closing surgical incisions. The camera or sensor may be a navigation camera or other appropriate sensor for viewing the surgical field and any markers present in the surgical field.

The third robotic arm holding the camera or sensor may be a dedicated surveillance configured to position a conventional surgical robotic navigation camera to view the suture alignment markers in a manner similar to viewing conventional registration and navigational markers. In other embodiments, the cameras or other sensors may be mounted on a surgical robotic arm capable of being positioned very closely to the incision to view the alignment markers, such as those described in commonly owned PCT Application WO2023/248005, the full disclosure of which has been previously incorporated herein by reference. In other sewing applications of the disclosed technology, the camera or other sensor may be held in a position to gain a view of a larger surgical field, such as for example a large section of a patient's spine wherein the various layers of tissue need to be sewn.

The surgical robotic arms may be positioned on a single cart or chassis that comprises a robotic controller configured to kinematically position the surgical robotic arms. For use in spinal and other orthopedic procedures, a mobile single cart or chassis can be positioned under a surgical table to bilaterally position the first and second robotic arms and needle holders on opposite sides of the surgical table.

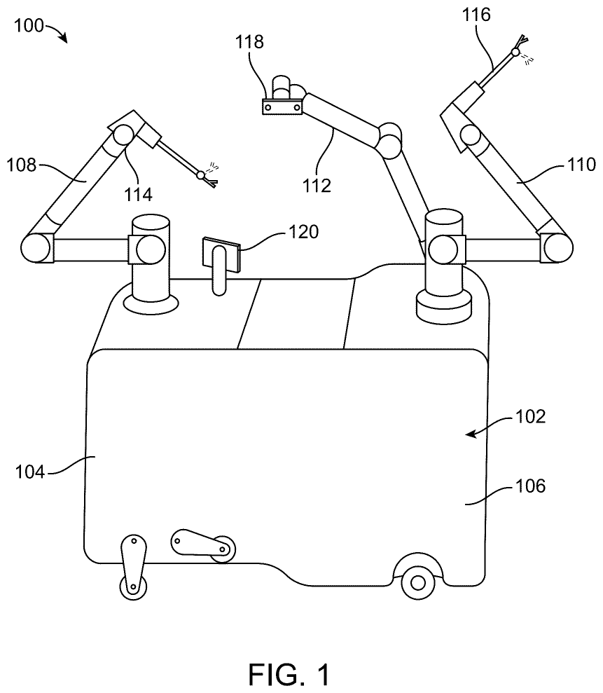

An exemplary robotic surgical system 100 intended particularly for use in the methods of the disclosed technology is shown in FIG. 1. The robotic surgical system 100 comprises a chassis 102, typically a single, rigid frame which provides a base or platform for three robotic arms 108, 110 and 112 that are placed relatively far apart on the chassis on opposite longitudinal ends 104 and 106 thereof, typically approximately one meter apart, thus allowing for desirable attributes such as reachability, maneuverability, and an ability to apply significant force. In the illustrated embodiment, robotic surgical arm 108 is on the first end 104 of the chassis 102 and robotic surgical arms 110 and 112 are on the second end 106 of the chassis. The chassis is preferably but not necessarily mobile, e.g. being in the form of a mobile cart as described in commonly owned WO2022/195460, previously incorporated herein by reference. In other embodiments and implementations, the surgical arms 108, 110 and 112 could be mounted on a base or other structure of a surgical table. For performing surgical suturing and sewing in accordance with the disclosed technology, it is necessary only that the robotic surgical arms be located on a stable platform that allows the arms to be moved within a common robotic coordinate system under the control of a surgical robotic controller, typically an on-board controller having a user interface, such as display screen 120.

The single, rigid chassis of the disclosed technology will usually comprise, consist of, or consist essentially of a single mobile cart, as disclosed for example in commonly owned WO2022/195460, the full disclosure of which has been previously incorporated herein by reference. In other instances, however, the single, rigid chassis may comprise separate modules, platforms, or components, that are assembled at or near the surgical table, as described for example in commonly owned PCT Application PCT/EP2024/052353, entitled Integrated Multi-Arm Mobile Surgical Robotic System, filed on Jan. 29, 2024, the full disclosure of which is incorporated herein by reference. The only requirement of the single, rigid chassis is that it provide a stable base for all the surgical arms so that they may be accurately and precisely kinematically positioned and tracked by the surgical robotic controller in a single surgical robotic coordinate space.

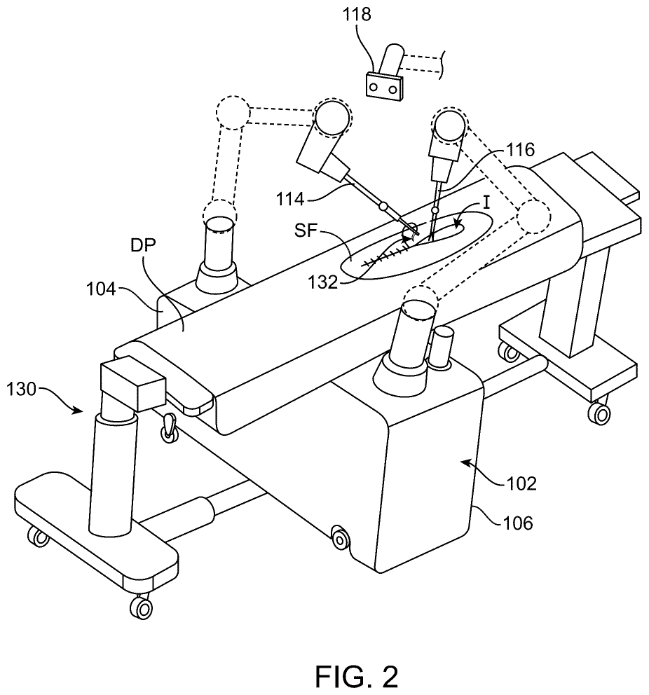

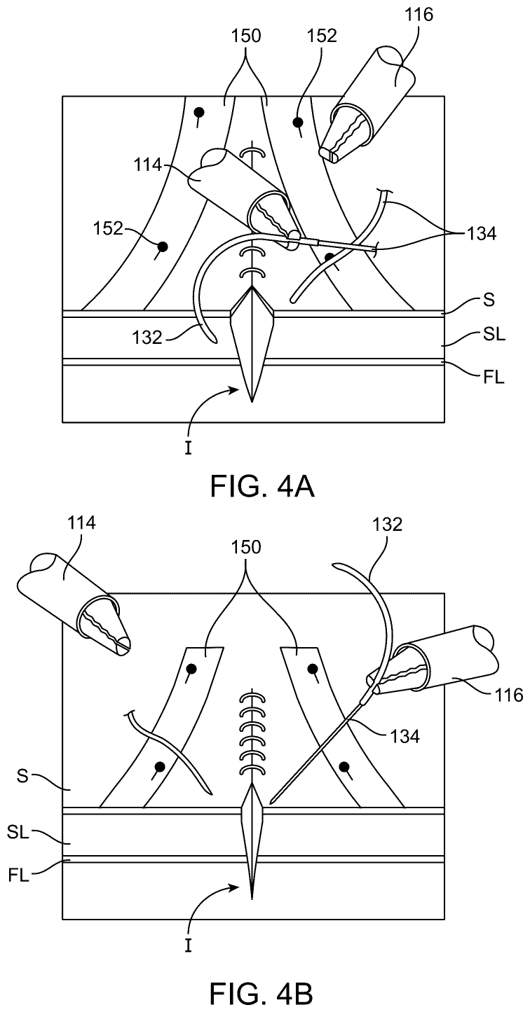

As shown in FIG. 2, the chassis 102 of the robotic surgical system 100 is typically configured to be temporarily placed under a surgical table 130 when performing a robotic surgical procedure, allowing the robotic surgical system 100 to be stored remotely before and after the procedure. The robotic arms 108, 110, and 112 may optionally be configured to be retracted into the chassis 102 of the robotic surgical system 100, allowing the system to be moved into or out of the surgical field in a compact configuration. The first and second surgical robotic arms 108 and 110 carry the first and second needle holders 114 and 116, respectively, and the needle holders are each configured to selectively grasp and release a surgical needle 132 (best seen in FIGS. 4A and 4B). A draped patient (DP) lies prone on the surgical table 130, and a midline incision (I) over the patient's spine is disposed in a surgical field SF exposed through the draping. The first and second surgical robotic arms 108 and 110 manipulate the first and second needle holders 114 and 116 to suture the incision (I) as will be described in greater detail below.

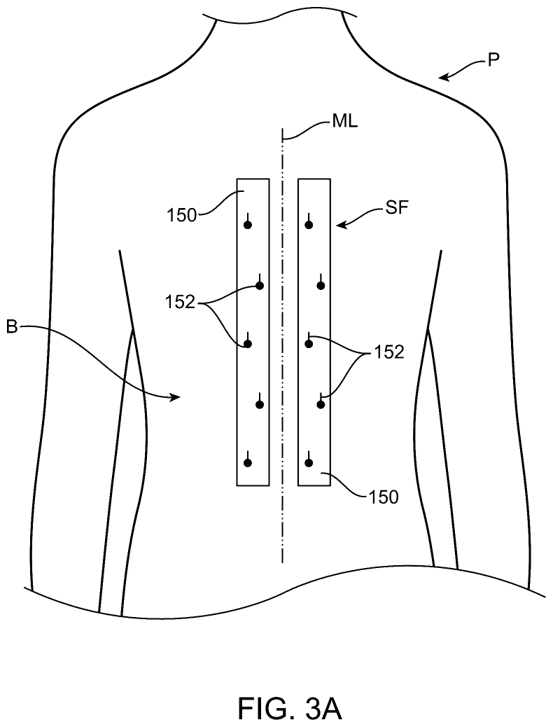

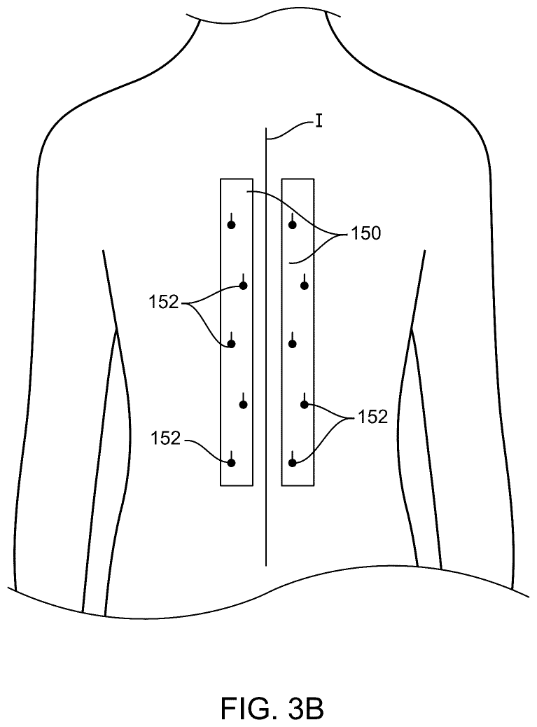

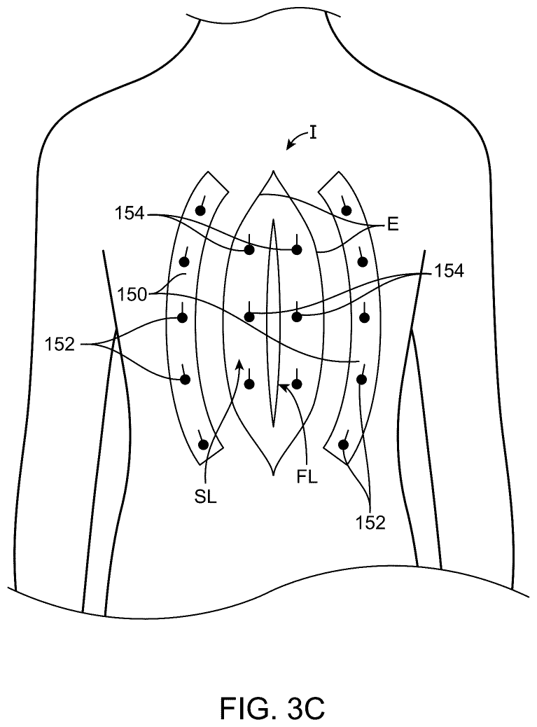

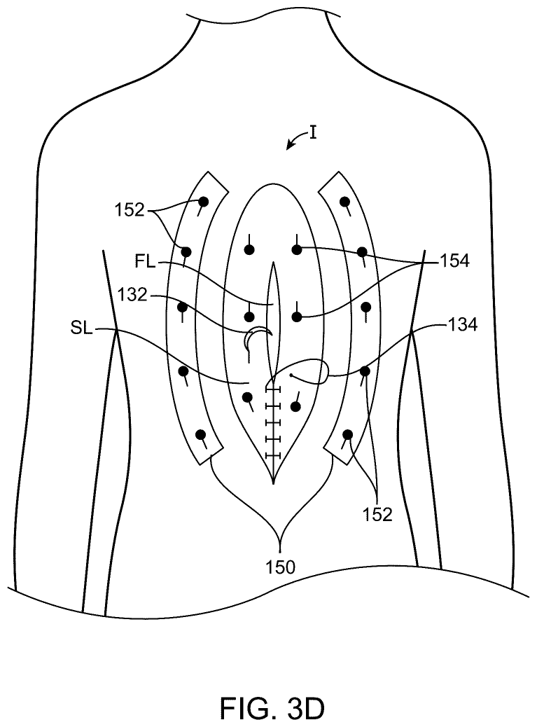

Referring now to FIGS. 3A to 3D, a spinal procedure may be performed with a patient P lying prone on the surgical table 130 with the surgical field SF exposed through draping (FIG. 2). Self-adhesive or other marker strips 150 are positioned on the skin of patient's back on opposite sides of the intended location of a midline incision to be used, for example, to expose the patient's spine for a robotic surgical spinal procedure. Each marker strip 150 will typically have a plurality of individual marker elements 152 arranged in a preselected pattern thereon. In many instances, each strip 150 will have an identical or mirror-image pattern, allowing the marker strips 150 to be placed so that the individual marker elements 152 align transversely across the patient's midline (ML). The strips 150 will typically be flexible to accommodate stretching of the back tissue during the procedure. The individual marker elements will be visible to the camera 118 or other sensor, typically being optically visible to an optical camera. Other markers and sensors could be used, however, such as radiopaque markers with fluoroscopic sensors, electromagnetically (EM) detectable markers with EM detectors, and the like. Also, individual markers could be placed on the patient's back without being supported on a strip or other carrier substrate.

While the individual markers are usually arranged in predetermined patterns, as illustrated in FIGS. 3A to 3D, in other instances the patterns could be random, either placed manually or by the surgical robot. After placement, such “random” patterns can be scanned by the surgical robotic system, for example using the camera 118 or other sensor, and the pattern saved in the surgical robotic controller as a baseline for comparison to further scans performed during the procedure to track changes in the soft tissues while sewing to close the incision.

As shown in FIG. 3B, after the marker strips 150 have been placed on the patient's back, an incision (I) can be made between the marker strips to expose the patient's spine.

As shown in FIG. 3C, after the incision (I) is formed, the opposed edges (E) can be retracted to fully expose the patient's spine. Retraction further exposes the patient's subcutaneous layer (SL) and fascial layer (FL). Additional individual or other markers 154 can optionally be placed directly on the subcutaneous layer (SL) (shown) and/or on the fascial layer (FL) (not shown). The markers 154 will often be biodegradable so that they do not need to be removed before the incision (I) is closed. For example, surgical inks may be placed by the user or the surgical robot directly on the exposed tissue without the need for removal. As with the markers 152 on the skin, the subcutaneously placed markers 154 may be arranged in a predetermined pattern or may be placed in a random pattern that is scanned by the surgical robotic system 100 to establish a baseline.

After the spinal procedure has been completed, the robotic surgical system 100 can be used to manipulate the surgical needle 132 to close the incision (I), as shown in FIGS. 3D, 4A and 4B. FIG. 3D shows generally that the surgical needle can be used to first close the fascial layer (FL) with suture 134, typically suture wire when closing incisions in the major muscles.

Details of using the first and second needle holders 114 and 116 of the robotic surgical system 100 for closing the incision (I) are best seen in FIGS. 4A and 4B. As seen in FIG. 4A, the first robotic surgical arm 108 (FIGS. 1 and 2), moves the first needle holder 114 to advance the surgical needle 132 through the patient's skin S and into the subcutaneous layer (SL) on one side of the incision (I). The first robotic surgical arm 108 may be controlled by the user through the display/controller 120 or in some cases by a remote controller interface. Alternatively, movement of the first robotic surgical arm 108 may be controlled primarily in an automatic mode by a robotic controller.

In either case, the robotic controller will monitor advancement of the needle holder 114 and displacement of markers 152 and/or 154 using the camera 118 or other sensor to determine if and how the soft tissue is being deformed. For example, the camera 118 or other sensor may detect displacement or misalignment of markers 152 or 154 which were transversely aligned before the beginning of the surgical procedure. In response to any detected misalignment, the robotic controller can kinematically or otherwise adjust movement of the first needle holder 114 to move the soft tissue back to its initial alignment by realigning the markers.

As shown in FIG. 4B, after the surgical needle 132 has been advanced fully across the incision (I), the surgical needle can be captured by the second needle holder 116 which is being manipulated by the second robotic surgical arm 110. The robotic controller 120 (either under primary user control or fully automatic control as described previously) uses the second robotic surgical arm 110 to draw the suture 134 away from the incision (I) while continuing to monitor the markers 152 and 154. As displacement or misalignment of the markers is detected, the robotic controller can adjust movement of the second robotic surgical arm 110, and in turn the needle holder 116, to offset misalignment of the markers and to minimize distortions of the soft tissue.

Such movement adjustments of the robotic surgical arms 108 and 110 can be accomplished kinematically, under feedback control based on the optical scanning, or by a combination of both approaches. In some instances, as the alignment is being optically or otherwise monitored by the camera 118 or other sensors, feedback control will usually be sufficient.

Reference numbers and letters used herein are listed in TABLE I below:

TABLE I
KEY TO REFERENCE NUMERALS AND LETTERS

| | |
|---|---|
| 100 | Robotic surgical system |
| 102 | Chassis |
| 104 | First end |
| 106 | Second end |
| 108 | Robotic surgical arm (first) |
| 110 | Robotic surgical arm (second) |
| 112 | Robotic surgical arm (third) |
| 114 | First needle holder |
| 116 | Second needle holder |
| 118 | Camera |
| 120 | Display/Controller |
| 130 | Surgical table |
| 132 | Surgical needle |
| 134 | Suture |
| 150 | Marker strip |
| 152 | Marker elements on strips |
| 154 | Marker elements on exposed |
| | tissue |
| B | Patient's back |
| DP | Draped patient |
| E | Edges of the incision |
| FL | Fascial layer |
| I | Incision |
| ML | Patient's midline |
| P | Patient |
| S | Patient's skin |
| SF | Surgical field |
| SL | Subcutaneous layer |

One of skill in the art will realize that several variations on the disclosed embodiments are possible while staying within the bounds of the current invention. Solely by way of example, different variations in the number of navigation cameras, robotic arms, markers and/or labels and end effectors can be used without departing from the invention. As another example, markers and labels of varying sizes, shapes and patterns can be used. As yet another example, numerous variations of surgical tools and surgical approaches to bilateral robotic sewing can be employed without departing from the invention described herein. The embodiments provided are representative in nature.

1.-19. (canceled)

20. A method for sewing an incision in a patient's soft tissue using a surgical robot having a surgical robotic controller and first and second surgical robotic arms, said method comprising the following steps performed by the surgical robotic controller:
(a) controlling movement of the first and second surgical robotic arms, with user input or automatically, to pass a surgical needle through opposite edges of the incision, wherein the surgical needle pulls a suture across the incision;
(b) controlling movement of the first and second surgical robotic arms, with user input or automatically, to tension the suture to close the opposite edges together;
(c) tracking positions of a plurality of markers placed on patient tissue on each side of the incision automatically as the suture is being tensioned; and
(d) adjusting movements of the first and second surgical robotic arms automatically in response to changes in tissue marker positions to properly align the opposite edges of the incision as the suture is being tensioned.

21. The method of claim 20, wherein controlling movement of the first and second surgical robotic arms to pass the surgical needle through opposite edges of the incision comprises the user inputting instructions to the surgical robotic controller via an interface to guide movement of the first and second surgical robotic arms.

22. The method of claim 21, wherein controlling movement of the first and second surgical robotic arms to tension the suture to close the opposite edges together comprises the user inputting instructions to the surgical robotic controller via the interface to guide movement of the first and second surgical robotic arms.

23. The method of claim 20, wherein at least some of the plurality of markers are located on the patient's skin on either side of the incision.

24. The method of claim 20, wherein at least some of the plurality of markers are located on opposed surfaces of the patient's soft tissue exposed by the incision.

25. The method of claim 20, wherein at least some of the plurality of markers are located on opposed surfaces of the patient's fascia exposed by the incision.

26. The method of claim 20, wherein at least some of the plurality of markers are placed on the patient's skin prior to making the incision.

27. The method of claim 20, wherein at least some of the plurality of markers are placed on the patient's skin after making the incision.

28. The method of claim 20, wherein at least some of the plurality of markers are two-dimensional.

29. The method of claim 28, wherein at least some of the two-dimensional markers comprise flexible stickers or a flexible ribbon that can be adhesively attached to the patient tissue.

30. The method of claim 28, wherein at least some of the two-dimensional markers are marked on the patient's tissue with an ink.

31. The method of claim 30, wherein the user manually marks the patient's tissue with a marking pen.

32. The method of claim 30, wherein the surgical robotic controller positions a surgical robotic arm to mark the patient's tissue with the ink.

33. The method of claim 20, wherein at least some of the plurality of markers are three-dimensional.

34. The method of claim 20, further comprising tracking positions of the plurality of markers placed on patient tissue on each side of the incision automatically as the surgical needle is passed through the opposite edges and adjusting movements of the first and second surgical robotic arms automatically in response to changes in tissue marker positions to properly align the opposite edges of the incision as the surgical needle is passed through said opposite edges.

35. The method of claim 20, wherein tracking positions of the plurality of markers placed on patient tissue on each side of the incision comprises scanning the tissue with a sensor controlled by the surgical robotic controller.

36. The method of claim 35, wherein scanning the tissue with a sensor controlled by the surgical robotic controller comprises controlling movement of a third surgical robotic arm to position a camera held by the third surgical robotic arm to optically scan the plurality of markers.

37. The method of claim 36, wherein tracking the positions of the plurality of markers placed on patient tissue on each side of the incision comprises scanning the tissue with the sensor prior to tensioning the suture to close the opposite edges together to establish a baseline marker pattern from which changes can be determined.

38. The method of claim 36, wherein tracking the positions of the plurality of markers placed on patient tissue on each side of the incision comprises scanning the tissue with the sensor prior to passing the surgical needle through opposite edges of the incision to establish a baseline marker pattern from which changes can be determined.

39. The method of claim 36, wherein tracking the positions of the plurality of markers placed on patient tissue on each side of the incision comprises scanning the tissue with the sensor prior to forming the incision to establish a baseline marker pattern from which changes can be determined.

40. The method of claim 39, further comprising scanning the patient's tissue with the sensor after the suture has been tensioned to confirm that the markers on the opposite edges of the incision remain properly aligned.

41.-55. (canceled)
